(19) United States
(12) Patent Application Publication
(10) Pub. No.: US 2024/0287584 A1
(43) Pub. Date: Aug. 29, 2024

(54) METHODS FOR PERFORMING TEMPERATURE MULTIPLEXED PCR WITH INCREASED SENSITIVITY

(71) Applicant: Roche Molecular Systems, Inc., Pleasanton, CA (US)

(72) Inventor: Ronald Kurnik, Foster City, CA (US)

(21) Appl. No.: 18/572,893

(22) PCT Filed: Jun. 24, 2022
(86) PCT No.: PCT/EP2022/067310

§ 371 Date: Dec. 21, 2023

Related U.S. Application Data
(60) Provisional application No. 62/215,137, filed on Sep. 7, 2015.

(51) Int. Cl. C12Q 1/682; C12Q 1/686

(52) U.S. Cl. CPC C12Q 1/682; C12Q 1/686

(57) ABSTRACT

The present invention describes methods for temperature multiplexed PCR for detection and quantitation of target nucleic acids with increased sensitivity. The methods are performed by contacting the sample with probes designed for temperature multiplexed PCR, measuring the fluorescence at multiple temperatures, subtracting signals to obtain calculated signals for each target of interest. Furthermore, the methods comprise obtaining a baseline of the first fluorescent signal, and adjusting the calculated signals using the obtained baseline of the first fluorescent signal to obtain a baseline shifted calculated signals.

### FIELD OF THE INVENTION

The present invention relates to methods for polymerase chain reaction (PCR) particularly to methods for performing temperature multiplexed PCR with increased sensitivity.

### BACKGROUND OF THE INVENTION

The polymerase chain reaction (PCR) has become a ubiquitous tool of biomedical research, disease monitoring and diagnostics. Amplification of nucleic acid sequences by PCR is described in U.S. Pat. Nos. 4,683,195, 4,683,202, and 4,965,188. PCR is now well known in the art and has been described extensively in the scientific literature. See PCR Applications, ((1999) Innis et al., eds., Academic Press, San Diego), PCR Strategies, ((1995) Innis et al., eds., Academic Press, San Diego); PCR Protocols, ((1990) Innis et al., eds., Academic Press, San Diego), and PCR Technology, ((1989) Erlich, ed., Stockton Press, New York). A “real-time” PCR assay is able to simultaneously amplify and detect and quantify the starting amount of the target sequence. The basic TaqMan real-time PCR assay using the 5′-to-3′ nuclease activity of the DNA polymerase is described in Holland et al., (1991) Proc. Natl. Acad. Sci. 88:7276-7280 and U.S. Pat. No. 5,210,015. The real-time PCR without the nuclease activity (a nuclease-free assay) has been described in a U.S. application Ser. No. 12/330,694 filed on Dec. 9, 2008. The use of fluorescent probes in real-time PCR is described in U.S. Pat. No. 5,538,848.

A typical real-time PCR protocol with fluorescent probes involves the use of a labeled probe, specific for each target sequence. The probe is preferably labeled with one or more fluorescent moieties, which absorb and emit light at specific wavelengths. Upon hybridizing to the target sequence or its amplicon, the probe exhibits a detectable change in fluorescent emission as a result of probe hybridization or hydrolysis.

The major challenge of the real-time assay however remains the ability to analyze numerous targets in a single tube. In virtually every field of medicine and diagnostics, the number of loci of interest increases rapidly. For example, multiple loci must be analyzed in forensic DNA profiling, pathogenic microorganism detection, multi-locus genetic disease screening and multi-gene expression studies, to name a few.

With the majority of current methods, the ability to multiplex an assay is limited by the detection instruments. Specifically, the use of multiple probes in the same reaction requires the use of distinct fluorescent labels. To simultaneously detect multiple probes, an instrument must be able to discriminate among the light signals emitted by each probe. The majority of current technologies on the market do not permit detection of more than four to seven separate wavelengths in the same reaction vessel. Therefore, using one uniquely-labeled probe per target, no more than four to seven separate targets can be detected in the same vessel. In practice, at least one target is usually a control nucleic acid. Accordingly, in practice, no more than three to six experimental targets can be detected in the same tube. The use of fluorescent dyes is also limited due to the spectral width where only about six or seven dyes can be fit within the visible spectrum without significant overlap interference. Thus the ability to multiplex an assay will not keep pace with the clinical needs, unless radical changes in the amplification and detection strategy are made.

In order to address the above needs, methods for detection of multiple target nucleic acids using temperature multiplexed PCR techniques have been disclosed in U.S. patent application Ser. No. 15/705,821 (published as US2018-073064A1), the content of which is hereby incorporated by reference in its entirety for all purposes. While these methods drastically improve the scope of multiplexing, in order to be clinically valuable, there is still a need to improve the sensitivity of such methods.

### SUMMARY OF THE INVENTION

The present invention provides for novel methods for nucleic acid sequence detection with improved sensitivity, particularly detection of multiple target nucleic acids using temperature multiplexing PCR techniques. The methods are performed by contacting the sample with probes designed for temperature multiplexed PCR, measuring the fluorescence at multiple temperatures, subtracting signals to obtain calculated signals for each target of interest, adjusting the baseline of the calculated signals, and evaluating the resultant signals to identify and quantify the targets.

In an aspect, a method for detecting two or more target nucleic acid sequences in a sample is provided. The method includes contacting the sample suspected of containing said two or more target nucleic acid sequences in a single reaction vessel with a pair of probes each comprising a common fluorescent dye, wherein a first probe of the pair of probes comprises a nucleotide sequence at least partially complementary to a first target nucleic acid sequence and a second probe of the pair of probes comprises a nucleotide sequence at least partially complementary to a second target nucleic acid sequence. The first and second probes may be designed for temperature multiplexed PCR.

The method further includes amplifying the first and second target nucleic acid sequences by polymerase chain reaction (PCR), measuring a first fluorescent signal of the common fluorescent dye at a first temperature, increasing temperature to a second temperature, which is higher than the first temperature, and measuring a second fluorescent signal of the common fluorescent dye at the second temperature. The method further includes subtracting the first fluorescent signal from the second fluorescent signal to obtain a calculated signal, obtaining a baseline of the first fluorescent signal, and adjusting the calculated signal using the obtained baseline of the first fluorescent signal to obtain a baseline shifted calculated signal.

The method further includes repeating the above steps in multiple PCR cycles to produce a desired quantity of amplification products from the first and second target nucleic acid sequences, and determining the presence of the first target nucleic acid sequence from the first fluorescent signals from the multiple PCR cycles and the presence of the second target nucleic acid sequence from the baseline shifted calculated signals from the multiple PCR cycles.

In some embodiments, obtaining the baseline of the first fluorescent signal may include performing non-linear regression analysis of the first fluorescent signal at the first temperature to identify an approximated signal curve and obtaining the baseline from the approximated signal curve. In some embodiments, obtaining the baseline of the first fluorescent signal may include determining a y-intercept of the approximated signal curve and adjusting the calculated signal may include matching a y-intercept of the calculated signal to the y-intercept of the approximated signal curve. In other embodiments, obtaining the baseline of the first fluorescent signal may include determining a y-intercept and a baseline slope of the approximated signal curve and adjusting the calculated signal may include matching a y-intercept of the calculated signal to the y-intercept of the approximated signal curve and matching a baseline slope of the calculated signal to the baseline slope of the approximated signal curve.

In some embodiments, determining the presence of the first target nucleic acid and the second target nucleic acid may include determining a series of relative fluorescence intensity values of the first fluorescent signals and a series of relative fluorescence intensity values of the baseline shifted calculated signals. For example, determining the presence of the first target nucleic acid sequence may include comparing the series of relative fluorescence intensity values of the first fluorescent signals to a threshold and determining the presence of the second target nucleic acid sequence may include comparing the series of relative fluorescence intensity values of the baseline shifted calculated signals to a second threshold. For instance, determining the presence of the first and second target nucleic acid may include determining a relative fluorescence intensity endpoint (RFIe) calculated based on the formula:

$$RFIe = \frac{last fluorescent value}{m edian [ first five fluorescent values ]}$$

In some embodiments, the method detects at least three target nucleic acid sequences in the sample. For example, the sample may also be contacted with a third probe comprising the common fluorescent dye and a nucleotide sequence at least partially complementary to a third target nucleic acid sequence and the third nucleic acid is also amplified by polymerase chain reaction (PCR). Such methods may further include increasing temperature to a third temperature, which is higher than the second temperature, measuring a third fluorescent signal of the common fluorescent dye at the third temperature, subtracting the second fluorescent signal from the third fluorescent signal to obtain a second calculated signal, and adjusting the second calculated signal using the obtained baseline of the first fluorescent signal to obtain a second baseline shifted calculated signal. The method may further include repeating the steps above in multiple PCR cycles to produce desired quantity of amplification products from the first, second, and third target nucleic acid sequences, and determining the presence of the third target nucleic acid sequence from the second baseline shifted calculated signals from the multiple PCR cycles.

### BRIEF DESCRIPTION OF THE DRAWINGS

FIG. 1 is a graphical representation of temperature multiplexing using a fluorescent probe at two temperatures in accordance with one embodiment of the methods of the present invention.

FIG. 2 is a graphical representation of temperature multiplexing at three temperatures in accordance with an embodiment of the methods of the present invention.

FIG. 3 shows the fluorescence signals at two temperatures and the subtracted signal without baseline matching.

FIG. 4 shows the fluorescence signals at the two temperatures shown in FIG. 3 and the baseline matched subtracted signal in accordance with an embodiment of the methods of the present disclosure.

FIG. 5 shows a plot of resultant RFIe values without baseline matching.

FIG. 6 shows a plot of resultant RFIe values using baseline matched signals in accordance with an embodiment of the methods of the present disclosure.

FIG. 7 shows a method for performing temperature multiplexed PCR with increased sensitivity using baseline matching in accordance with an embodiment of the methods of the present disclosure.

### DETAILED DESCRIPTION OF THE INVENTION

### Definitions

The term “sample” as used herein includes a specimen or culture (e.g., microbiological cultures) that includes nucleic acids. The term “sample” is also meant to include both biological and environmental samples. A sample may include a specimen of synthetic origin. Biological samples include whole blood, serum, plasma, umbilical cord blood, chorionic villi, amniotic fluid, cerebrospinal fluid, spinal fluid, lavage fluid (e.g., bronchioalveolar, gastric, peritoneal, ductal, ear, arthroscopic), biopsy sample, urine, feces, sputum, saliva, nasal mucous, prostate fluid, semen, lymphatic fluid, bile, tears, sweat, breast milk, breast fluid, embryonic cells and fetal cells. In a preferred embodiment, the biological sample is blood, and more preferably plasma. As used herein, the term “blood” encompasses whole blood or any fractions of blood, such as serum and plasma as conventionally defined. Blood plasma refers to the fraction of whole blood resulting from centrifugation of blood treated with anticoagulants. Blood serum refers to the watery portion of fluid remaining after a blood sample has coagulated. Environmental samples include environmental material such as surface matter, soil, water and industrial samples, as well as samples obtained from food and dairy processing instruments, apparatus, equipment, utensils, disposable and non-disposable items. These examples are not to be construed as limiting the sample types applicable to the present invention.

The terms “target” or “target nucleic acid” as used herein are intended to mean any molecule whose presence is to be detected or measured or whose function, interactions or properties are to be studied. Therefore, a target includes essentially any molecule for which a detectable probe (e.g., oligonucleotide probe) or assay exists, or can be produced by one skilled in the art. For example, a target may be a biomolecule, such as a nucleic acid molecule, a polypeptide, a lipid, or a carbohydrate, which is capable of binding with or otherwise coming in contact with a detectable probe (e.g., an antibody), wherein the detectable probe also comprises nucleic acids capable of being detected by methods of the invention. As used herein, “detectable probe” refers to any molecule or agent capable of hybridizing or annealing to a target biomolecule of interest and allows for the specific detection of the target biomolecule as described herein. In one aspect of the invention, the target is a nucleic acid, and the detectable probe is an oligonucleotide. The terms “nucleic acid” and “nucleic acid molecule” may be used interchangeably throughout the disclosure. The terms refer to oligonucleotides, oligos, polynucleotides, deoxyribonucleotide (DNA), genomic DNA, mitochondrial DNA (mtDNA), complementary DNA (cDNA), bacterial DNA, viral DNA, viral RNA, RNA, message RNA (mRNA), transfer RNA (tRNA), ribosomal RNA (rRNA), siRNA, catalytic RNA, clones, plasmids, M13, P1, cosmid, bacteria artificial chromosome (BAC), yeast artificial chromosome (YAC), amplified nucleic acid, amplicon, PCR product and other types of amplified nucleic acid, RNA/DNA hybrids and polyamide nucleic acids (PNAs), all of which can be in either single- or double-stranded form, and unless otherwise limited, would encompass known analogs of natural nucleotides that can function in a similar manner as naturally occurring nucleotides and combinations and/or mixtures thereof. Thus, the term “nucleotides” refers to both naturally-occurring and modified/nonnaturally-occurring nucleotides, including nucleoside tri, di, and monophosphates as well as monophosphate monomers present within polynucleic acid or oligonucleotide. A nucleotide may also be a ribo; 2′-deoxy; 2′,3′-deoxy as well as a vast array of other nucleotide mimics that are well-known in the art. Mimics include chain-terminating nucleotides, such as 3′-O-methyl, halogenated base or sugar substitutions; alternative sugar structures including nonsugar, alkyl ring structures; alternative bases including inosine; deaza-modified; chi, and psi, linker-modified; mass label-modified; phosphodiester modifications or replacements including phosphorothioate, methylphosphonate, boranophosphate, amide, ester, ether; and a basic or complete internucleotide replacements, including cleavage linkages such a photocleavable nitrophenyl moieties.

The presence or absence of a target can be measured quantitatively or qualitatively. Targets can come in a variety of different forms including, for example, simple or complex mixtures, or in substantially purified forms. For example, a target can be part of a sample that contains other components or can be the sole or major component of the sample. Therefore, a target can be a component of a whole cell or tissue, a cell or tissue extract, a fractionated lysate thereof or a substantially purified molecule. Also a target can have either a known or unknown sequence or structure.

The term “amplification reaction” refers to any in vitro means for multiplying the copies of a target sequence of nucleic acid.

“Amplifying” refers to a step of submitting a solution to conditions sufficient to allow for amplification. Components of an amplification reaction may include, but are not limited to, e.g., primers, a polynucleotide template, polymerase, nucleotides, dNTPs and the like. The term “amplifying” typically refers to an “exponential” increase in target nucleic acid. However, “amplifying” as used herein can also refer to linear increases in the numbers of a select target sequence of nucleic acid, but is different than a one-time, single primer extension step.

“Polymerase chain reaction” or “PCR” refers to a method whereby a specific segment or subsequence of a target double-stranded DNA, is amplified in a geometric progression. PCR is well known to those of skill in the art; see, e.g., U.S. Pat. Nos. 4,683,195 and 4,683,202; and PCR Protocols: A Guide to Methods and Applications, Innis et al., eds, 1990.

“Oligonucleotide” as used herein refers to linear oligomers of natural or modified nucleosidic monomers linked by phosphodiester bonds or analogs thereof. Oligonucleotides include deoxyribonucleosides, ribonucleosides, anomeric forms thereof, peptide nucleic acids (PNAs), and the like, capable of specifically binding to a target nucleic acid. Usually monomers are linked by phosphodiester bonds or analogs thereof to form oligonucleotides ranging in size from a few monomeric units, e.g., 3-4, to several tens of monomeric units, e.g., 40-60. Whenever an oligonucleotide is represented by a sequence of letters, such as “ATGCCTG,” it will be understood that the nucleotides are in 5′-3′ order from left to right and that “A” denotes deoxyadenosine, “C” denotes deoxycytidine, “G” denotes deoxyguanosine, “T” denotes deoxythymidine, and “U” denotes the ribonucleoside, uridine, unless otherwise noted. Usually oligonucleotides comprise the four natural deoxynucleotides; however, they may also comprise ribonucleosides or non-natural nucleotide analogs. Where an enzyme has specific oligonucleotide or polynucleotide substrate requirements for activity, e.g., single stranded DNA, RNA/DNA duplex, or the like, then selection of appropriate composition for the oligonucleotide or polynucleotide substrates is well within the knowledge of one of ordinary skill.

As used herein “oligonucleotide primer”, or simply “primer”, refers to a polynucleotide sequence that hybridizes to a sequence on a target nucleic acid template and facilitates the detection of an oligonucleotide probe. In amplification embodiments of the invention, an oligonucleotide primer serves as a point of initiation of nucleic acid synthesis. In non-amplification embodiments, an oligonucleotide primer may be used to create a structure that is capable of being cleaved by a cleavage agent. Primers can be of a variety of lengths and are often less than 50 nucleotides in length, for example 12-25 nucleotides, in length. The length and sequences of primers for use in PCR can be designed based on principles known to those of skill in the art.

The term “oligonucleotide probe” as used herein refers to a polynucleotide sequence capable of hybridizing or annealing to a target nucleic acid of interest and allows for the specific detection of the target nucleic acid.

A “reporter moiety” or “reporter molecule” is a molecule that confers a detectable signal. The detectable phenotype can be colorimetric, fluorescent or luminescent, for example. A “quencher moiety” or “quencher molecule” is a molecule that is able to quench the detectable signal from the reporter moiety.

A “mismatched nucleotide” or a “mismatch” refers to a nucleotide that is not complementary to the target sequence at that position or positions. An oligonucleotide probe may have at least one mismatch, but can also have 2, 3, 4, 5, 6 or 7 or more mismatched nucleotides.

The term “polymorphism” as used herein refers to an allelic variant. Polymorphisms can include single nucleotide polymorphisms (SNP's) as well as simple sequence length polymorphisms. A polymorphism can be due to one or more nucleotide substitutions at one allele in comparison to another allele or can be due to an insertion or deletion, duplication, inversion and other alterations known to the art.

The term “specific” or “specificity” in reference to the binding of one molecule to another molecule, such as a probe for a target polynucleotide, refers to the recognition, contact, and formation of a stable complex between the two molecules, together with substantially less recognition, contact, or complex formation of that molecule with other molecules. As used herein, the term “anneal” refers to the formation of a stable complex between two molecules.

A probe is “capable of annealing” to a nucleic acid sequence if at least one region of the probe shares substantial sequence identity with at least one region of the complement of the nucleic acid sequence. “Substantial sequence identity” is a sequence identity of at least about 80%, preferably at least about 85%, more preferably at least about 90%, 95% or 99%, and most preferably 100%. For the purpose of determining sequence identity of a DNA sequence and a RNA sequence, U and T often are considered the same nucleotide. For example, a probe comprising the sequence ATCAGC is capable of hybridizing to a target RNA sequence comprising the sequence GCUGAU.

The term “cleavage agent” as used herein refers to any means that is capable of cleaving an oligonucleotide probe to yield fragments, including but not limited to enzymes. For methods wherein amplification does not occur, the cleavage agent may serve solely to cleave, degrade or otherwise separate the second portion of the oligonucleotide probe or fragments thereof. The cleavage agent may be an enzyme. The cleavage agent may be natural, synthetic, unmodified or modified.

For methods wherein amplification occurs, the cleavage agent is preferably an enzyme that possesses synthetic (or polymerization) activity and nuclease activity. Such an enzyme is often a nucleic acid amplification enzyme. An example of a nucleic acid amplification enzyme is a nucleic acid polymerase enzyme such as Thermus aquaticus (Taq) DNA polymerase (TaqMan′) or E. coli DNA polymerase I. The enzyme may be naturally occurring, unmodified or modified.

A “nucleic acid polymerase” refers to an enzyme that catalyzes the incorporation of nucleotides into a nucleic acid. Exemplary nucleic acid polymerases include DNA polymerases, RNA polymerases, terminal transferases, reverse transcriptases, telomerases and the like.

A “thermostable DNA polymerase” refers to a DNA polymerase that is stable (i.e., resists breakdown or denaturation) and retains sufficient catalytic activity when subjected to elevated temperatures for selected periods of time. For example, a thermostable DNA polymerase retains sufficient activity to effect subsequent primer extension reactions, when subjected to elevated temperatures for the time necessary to denature double-stranded nucleic acids. Heating conditions necessary for nucleic acid denaturation are well known in the art and are exemplified in U.S. Pat. Nos. 4,683,202 and 4,683,195. As used herein, a thermostable polymerase is typically suitable for use in a temperature cycling reaction such as the polymerase chain reaction (“PCR”). The examples of thermostable nucleic acid polymerases include Thermus aquaticus Taq DNA polymerase, Thermus sp. Z05 polymerase, Thermus flavus polymerase, Thermotoga maritima polymerases, such as TMA-25 and TMA-30 polymerases, Tth DNA polymerase, and the like.

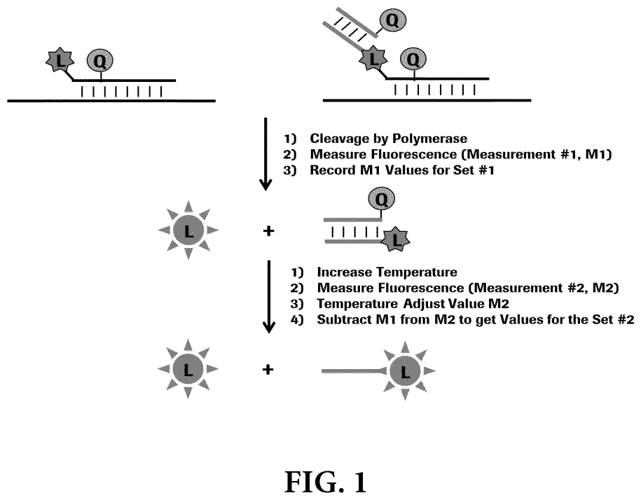

As described in the aforementioned U.S. patent application Ser. No. 15/705,821 (incorporated by reference herein), temperature multiplexed PCR techniques allow for each distinct fluorescent probe to be used to identify multiple targets by monitoring the fluorescence at distinct temperatures. The monitored signals at the lower temperatures may be subtracted from subsequent (higher) temperature signals to obtain the signal values of interest. (See FIG. 1). More specifically, as described in the aforementioned application, temperature multiplexing may be carried out by designing the probes to have tag portions hybridized to their respective quenching oligonucleotide molecules at various Tm temperatures, measuring the fluorescence at multiple temperatures at or above the Tm temperatures, and processing the resultant signals to identify and quantify the targets.

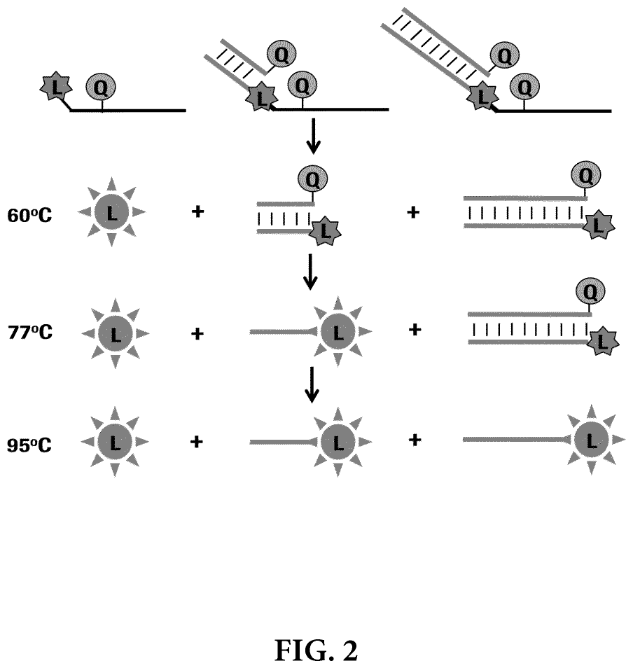

For example, amplification and detection of three target nucleic acid in one reaction can be achieved by using three oligonucleotide probes all labeled with the same fluorophore. A standard TaqMan® oligonucleotide probe may be used to detect the first target by measuring the fluorescent signal at a first temperature (usually the annealing temperature of a PCR cycle). A first “tagged” probe with a low Tm tag-quenching oligonucleotide duplex may be used to detect the second target by measuring the calculated fluorescent value at a second temperature at or above its Tm temperature and that is higher than the first temperature. A second “tagged” probe with a high Tm tag-quenching oligonucleotide duplex may be used to detect the third target by measuring the calculated fluorescent value at a third temperature at or above its Tm temperature and that is higher than the second temperature. (see FIG. 2) While this methodology theoretically enables the use of one TaqMan′ probe and two different tagged probes with four to seven different reporter moieties (e.g. fluorescent dyes) to detect between 12 and 21 different target nucleic acids in one reaction or one TaqMan′ probe and 3 different tagged probes to detect between 16 and 28 different target nucleic acids in one reaction, improved sensitivity is desired, as will be demonstrated in accordance with embodiments with the present disclosure.

In a typical case of a single fluorescent channel read at three temperatures T1, T2, and T3 (in ascending order for purposes of this example), where T1 is associated with Target “A”, T2 with Target “B”, and T3 with Target “C,” the signal of interest for Target “A” is simply the signal produced at T1, the signal of interest for Target “B” is the signal(T2)−signal(T1), and the signal for Target “C” is the signal(T3)−signal(T2). It will be understood that this signal subtraction may cause the signals of interest for Targets “B” and “C” to have different baseline intercepts than that of Target “A”. In practice, it has been found that the target sensitivity using these signals in this way, without further processing of the signals, is less than desired to be able to reliably and accurately identify the presence of and/or quantify the relevant targets.

The present invention provides for improved sensitivity by correcting the signals of interest that are calculated by subtraction of other signals. In the case of using a single fluorescent dye, e.g. FAM, with probes designed to hybridize to their respective quenching oligonucleotide molecules at differing temperatures such that three different targets can be evaluated by monitoring the fluorescent signal at three temperatures (T1<T2<T3), the signals for these three temperatures would be calculated as follows (where FAM(T) is the relative fluorescence of the FAM measured at a given temperature T):

$$Signal ( T 1 ) = FAM ( T 1 ) \qquad Eq . 1$$

$$Signal ( T 2 ) = FAM ( T 2 ) - ( FAM\_m1 ) \times FAM ( T 1 ) \qquad Eq . 2$$

$$Signal ( T 3 ) = FAM ( T 3 ) - ( FAM\_m2 ) \times FAM ( T 2 ) \qquad Eq . 3$$

In this case, FAM_m1 and FAM_m2 may be dye specific multipliers that are determined in order to equalize the gain between signal temperatures. For example, the multipliers may be calculated by evaluating Eq. 4 for each temperature signal and then solving for the variable using Eqs. 5 and 6.

$$mT = median [ \text{} last 5 fluorescence values ] \text{} - median [ first 5 fluorescence values ] . \qquad Eq . 4$$

$$xFAM ( T 1 ) = mT 2 / mT 1 \qquad Eq . 5$$

$$xFAM ( T 2 ) = mT 3 / mT 1 \qquad Eq . 6$$

Upon performing the above calculations, including the simple subtraction of signal values as described in Eqs. 2 and 3 to obtain the three relevant signals used in the identification and/or quantification of the three targets, it will be understood that each of these signals will necessarily have different baseline intercepts. In many instances, any processing of such baseline intercepts would not be suitable, because this would not maintain the scale invariance needed to properly evaluate the signals. However, for a given fluorescent dye in a single well, the optical path is identical at all temperatures. Thus, matching the baseline intercepts actually maintains the scale invariance since it is simply matching a given signal to that of the same fluorophore at a different temperature. Thus, in accordance with the present invention, a more accurate representation of the fluorescent signal at subsequent higher temperatures may be obtained by matching the baseline of signals at T2 and T3 with the baseline of the signal at T1. In some embodiments of the present invention, this baseline alignment or matching may be effected by matching the y-intercept of signals T2 and T3 with that of signal T1. In other embodiments, the baseline alignment or matching may be effected by matching both the y-intercept and baseline slope of signals T2 and T3 with those of signal T1. It will be understood that while it may be desirable and most accurate to match both the y-intercept and baseline slopes of the signals with those of signal T1, it may also be more computationally intensive. Accordingly, it may be suitable to simply match the y-intercepts, as this may sufficiently improve the sensitivity of subsequent calculations.

In accordance with the present invention, this may be done by identifying an approximation to a curve that fits the PCR dataset of the signal at the initial temperature, and then substituting the relevant baseline parameters of that approximation for the subsequent higher temperature signals. As described in commonly owned U.S. Pat. No. 7,680,868, which is hereby incorporated by reference in its entirety for all purposes, in many cases the PCR dataset is fit to an approximation using Equation 7, or a similar function that includes a baseline intercept plus slope, where x refers to the cycle number and z₁ to z₇ are adjustable parameters.

$$f ( x , \begin{matrix}r \\ z\end{matrix} ) = {z}_{1} + {z}_{2} x + \frac{{z}_{3}}{[ 1 + exp ( - {z}_{4} ( x - {z}_{5} ) ) ] [ 1 + exp ( - {z}_{6} ( x - {z}_{7} ) ) ]} \qquad Eq . 7$$

In this example, the y-intercept is given as z and the baseline slope is given as z₂. Thus, baseline matching of signal(T2) and signal(T3) with that of signal(T1) may be done by substituting z₁, or z₁+z₂ x of signal(T2), and signal(T3) with that determined from the approximation of signal (T1). As will be described below in further detail, such baseline matching greatly improves the target sensitivity, which improves the reliability and accuracy of determinations and quantifications made using the methods of the present invention.

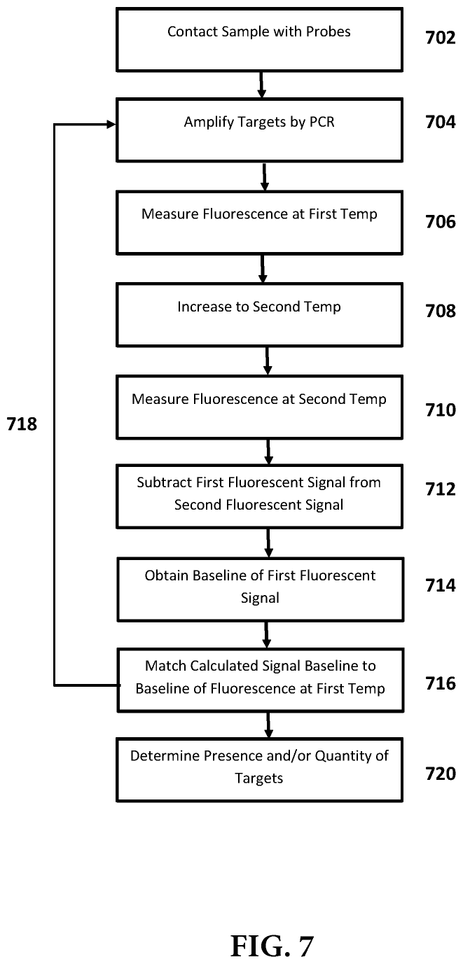

FIG. 7 shows a method for performing temperature multiplexed PCR with increased sensitivity using baseline matching in accordance with an embodiment of the methods of the present disclosure. In a first step 702, a sample suspected of containing the target nucleic acids is contacted inside a single reaction vessel (e.g. a single test tube or a single well in a multi-well microplate) with the probes designed for temperature multiplexed PCR. For example, the sample may be suspected of containing at least two distinct target nucleic acids. The probes may be designed for temperature multiplexed PCR by using a single fluorescent dye monitored at multiple temperatures. For example, the probes may be designed for temperature multiplexing by having tag portions hybridized to their respective quenching oligonucleotide molecules at various Tm temperatures. After being contacted with the probes, at step 704, the target nucleic acids are amplified by PCR.

Next, at step 706, the fluorescence signal is measured at a first temperature. For example, the first temperature may be the annealing and/or extension temperature of the PCR cycle. The signal measured at the first temperature may be indicative of the presence of the first target nucleic acid.

Next, at step 708, the temperature is gradually increased to a second temperature. The second temperature may be at or above the melting temperature associated with the tag-quenching oligonucleotide duplex of the second probe. At step 710, the fluorescence signal is measured at the second temperature. Then, in order to obtain the signal indicative of the presence of the second target nucleic acid, at step 712, a calculated signal value is determined by subtracting the signal detected at the first temperature from the signal detected at the second temperature. The calculated signal value may optionally be normalized for correction of signals that may be affected by temperature. For example, fluorescent signals are known to decrease at higher temperatures, and therefore, standards can be used to normalize the signal values obtained at different temperatures.

Optionally, if more than two targets are being evaluated, a further step (not shown in FIG. 7) may be provided where the temperature may be increased to a third temperature that is at or above the melting temperature associated with the tag-quenching oligonucleotide duplex of the third probe. The fluorescent signal may be measured at the third temperature, and a second calculated signal may be obtained by subtracting the fluorescent signal at the second temperature from the fluorescent signal at the third temperature.

At step 714, a baseline of the fluorescent signal at the first temperature is obtained. The baseline may include the y-intercept and/or the baseline slope of the fluorescent signal at the first temperature. The baseline may be obtained by any suitable method for obtaining such parameters of an evaluated signal. In some embodiments, the baseline may be obtained by performing nonlinear regression analysis of the measured signal. For example, the measured signal may be approximated using Eq. 7 and the baseline may be obtained by determining the y-intercept and/or baseline slope values of the approximated signal determined from the measured signal.

At step 716, the baseline of the calculated signal from step 712 is adjusted to match the baseline obtained in step 714. In some embodiments, only the y-intercept is used for matching the baseline. In some embodiments, both the y-intercept and the baseline slope are used for matching the baselines. If a second calculated signal is obtained (e.g. if three targets are being evaluated), the baseline of the second calculated signal may also be adjusted to match the baseline obtained in step 714.

As depicted by arrow 718, these signal measurements and calculations may be repeated and performed at multiple PCR cycles. Then, at step 720, once the desired number of cycles have been repeated, the determined cumulative signal values can be used to determine not only the presence or absence but also the quantity of the target nucleic acids. This may be done by determining the threshold value (Ct value) from a PCR growth curve generated from the signal values calculated plotted against PCR cycle number. For example, the RFIe may be determined using Eq. 8 described below, and a threshold value may be set based on the RFIe values.

Embodiments of the present invention will be further described in the following examples, which do not limit the scope of the invention described in the claims.

### EXAMPLES

### Example 1

A Real-time PCR study was conducted to evaluate the effect of baseline matching in accordance with embodiments of the present invention. The study was conducted using samples that contained various concentrations of Human Papillomavirus (HPV). The fluorescent probes used included Coumarin, FAM, HEX, JA270, and DKR. The fluorescence was evaluated at three temperatures, 58° C. (T1), 80° C. (T2), and 96° C. (T3). To understand the effect of baseline matching in accordance with embodiments of the present invention, the signals associated with FAM were evaluated and calculations were performed with and without baseline matching. Specifically, RFIe values, which are a measure of scale-invariant growth of a PCR curve that can be determined by Eq. 8 below, were determined with and without baseline matching.

$$RFIe = \frac{last fluorescent value}{m edian [ first five fluorescent values ]} \qquad Eq . 8$$

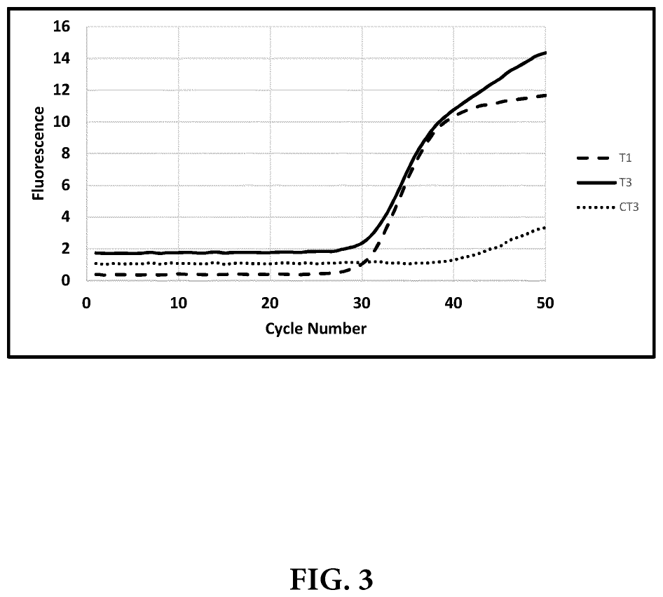

FIG. 3 shows the fluorescence signals at T1 and T3, and the calculated signal (CT3) using Eq. 3, where FAM_m2=0.91, without any baseline matching. It can be seen in FIG. 3 that the baseline of CT3 is not aligned with that of T1. RFIe values for the curves depicted in FIG. 3 are provided in the Table below:

| Signal | RFIe |
|---|---|
| FAM(T1) | 31.6211 |
| FAM(T2) | 6.8158 |
| FAM(T3) | 3.1355 |

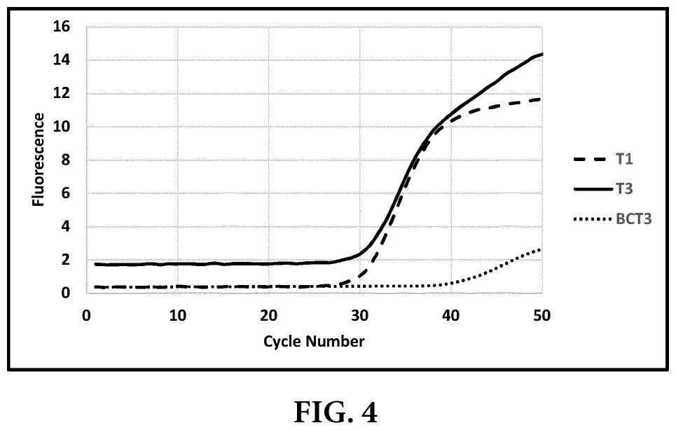

FIG. 4 shows the fluorescence signals at T1 and T3, and the baseline shifted calculated signal (BCT3). In this case, the baseline was shifted by substituting the y-intercept of CT3 with that of T1. It can be seen in FIG. 4 that the baseline of BCT3 is aligned with that of T1. RFIe values for the curves depicted in FIG. 4 are provided in the Table below:

| Signal | RFIe |
|---|---|
| FAM(T1) | 31.6211 |
| FAM(T2) [w/baseline matching] | 7.7803 |
| FAM(T3) [w/baseline matching] | 7.1557 |

It can be seen that the RFIe values for the curve associated with T3 significantly increases when baseline matching is employed. Thus, it is clear that baseline matching can have an important effect on the identification and quantification of the target.

### Example 2

A Real-time PCR study similar to Example 1, but with a larger data set was conducted to evaluate the sensitivity improvement of baseline matching.

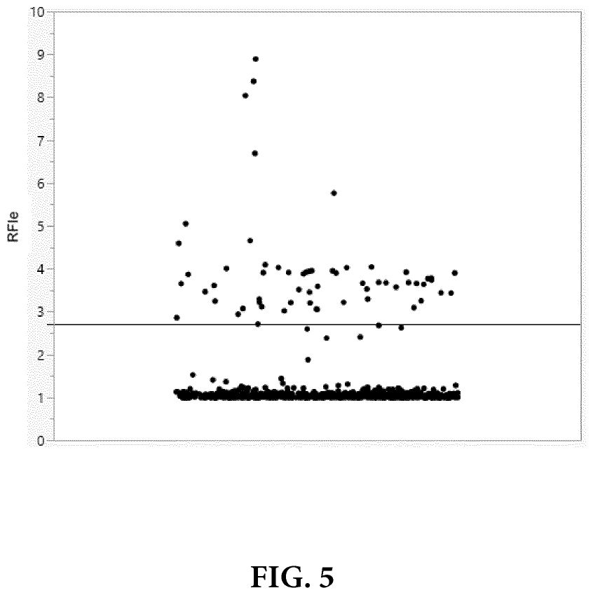

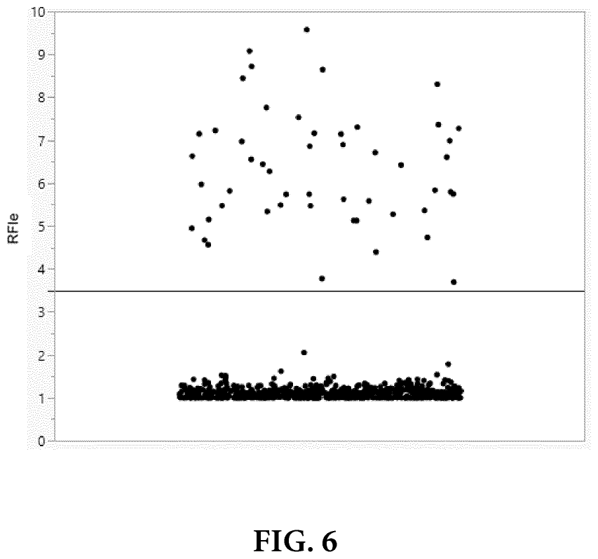

FIG. 5 shows a plot of resultant RFIe values of the data set without baseline matching, and FIG. 6 shows a plot of resultant RFIe values using baseline matched signals. In each case, a horizontal line depicts the separation of positive versus negative curve calls, i.e. determination of the presence of the targets. However, it can be seen that in FIG. 6, which has values calculated after using baseline matching in accordance with embodiments of the present invention, there is a much clearer separation between positive and negative curve calls. In contrast, it can be seen in FIG. 5, where baseline matching was not used, that there is simply not a clear demarcation between positive and negative curve calls. These results illustrate the improved sensitivity that can be obtained using baseline matching in accordance with embodiments of the present invention.

While the foregoing invention has been described in some detail for purposes of clarity and understanding, it will be clear to one skilled in the art from a reading of this disclosure that various changes in form and detail can be made without departing from the true scope of the invention. For example, the methods described above can be used in various combinations.

1. A method for detecting two or more target nucleic acid sequences in a sample comprising the steps of:
(a) contacting said sample suspected of containing said two or more target nucleic acid sequences in a single reaction vessel with a pair of probes each comprising a common fluorescent dye, wherein a first probe of the pair of probes comprises a nucleotide sequence at least partially complementary to a first target nucleic acid sequence and a second probe of the pair of probes comprises a nucleotide sequence at least partially complementary to a second target nucleic acid sequence, wherein the first and second probes are designed for temperature multiplexed PCR;
(b) amplifying the first and second target nucleic acid sequences by polymerase chain reaction (PCR);
(c) measuring a first fluorescent signal of the common fluorescent dye at a first temperature;
(d) increasing temperature to a second temperature, which is higher than the first temperature;
(e) measuring a second fluorescent signal of the common fluorescent dye at the second temperature;
(f) subtracting the first fluorescent signal from the second fluorescent signal to obtain a calculated signal;
(g) obtaining a baseline of the first fluorescent signal;
(h) adjusting the calculated signal using the obtained baseline of the first fluorescent signal to obtain a baseline shifted calculated signal;
(i) repeating steps (b) through (h) in multiple PCR cycles to produce a desired quantity of amplification products from the first and second target nucleic acid sequences;
(j) determining the presence of the first target nucleic acid sequence from the first fluorescent signals from the multiple PCR cycles and the presence of the second target nucleic acid sequence from the baseline shifted calculated signals from the multiple PCR cycles.

2. The method of claim 1, wherein obtaining the baseline of the first fluorescent signal comprises performing non-linear regression analysis of the first fluorescent signal at the first temperature to identify an approximated signal curve and obtaining the baseline from the approximated signal curve.

3. The method of claim 2, wherein obtaining the baseline of the first fluorescent signal comprises determining a y-intercept of the approximated signal curve.

4. The method of claim 3, wherein adjusting the calculated signal comprises matching a y-intercept of the calculated signal to the y-intercept of the approximated signal curve.

5. The method of claim 2, wherein obtaining the baseline of the first fluorescent signal comprises determining a y-intercept and a baseline slope of the approximated signal curve.

6. The method of claim 5, wherein adjusting the calculated signal comprises matching a y-intercept of the calculated signal to the y-intercept of the approximated signal curve and matching a baseline slope of the calculated signal to the baseline slope of the approximated signal curve.

7. The method of claim 1, wherein determining the presence of the first target nucleic acid and the second target nucleic acid comprises determining a series of relative fluorescence intensity values of the first fluorescent signals and a series of relative fluorescence intensity values of the baseline shifted calculated signals.

8. The method of claim 7, wherein:
(i) determining the presence of the first target nucleic acid sequence comprises comparing the series of relative fluorescence intensity values of the first fluorescent signals to a threshold; and
(ii) determining the presence of the second target nucleic acid sequence comprises comparing the series of relative fluorescence intensity values of the baseline shifted calculated signals to a second threshold.

9. The method of claim 7, wherein determining the presence of the first and second target nucleic acid comprises determining a relative fluorescence intensity endpoint (RFIe) calculated based on the formula:
$$RFIe = \frac{last fluorescent value}{m edian [ first five fluorescent values ]}$$

10. The method of claim 1, wherein at least three target nucleic acid sequences are detected in the sample, and wherein at step (a) the sample is also contacted with a third probe comprising the common fluorescent dye and a nucleotide sequence at least partially complementary to a third target nucleic acid sequence, and at step (b) the third nucleic acid is also amplified by polymerase chain reaction (PCR), the method further comprising the steps of:
(e1) after step (e), increasing temperature to a third temperature, which is higher than the second temperature;
(f1) measuring a third fluorescent signal of the common fluorescent dye at the third temperature;
(g1) subtracting the second fluorescent signal from the third fluorescent signal to obtain a second calculated signal;
(h1) adjusting the second calculated signal using the obtained baseline of the first fluorescent signal to obtain a second baseline shifted calculated signal;
(i1) repeating steps (b) through (h) and (e1) through (h1) in multiple PCR cycles to produce desired quantity of amplification products from the first, second, and third target nucleic acid sequences; and
(j1) determining the presence of the third target nucleic acid sequence from the second baseline shifted calculated signals from the multiple PCR cycles.

11. The method of claim 10, wherein obtaining the baseline of the first fluorescent signal comprises determining a y-intercept of the first fluorescent signal.

12. The method of claim 11, wherein adjusting the calculated signals each comprise matching a y-intercept of the calculated signals to the y-intercept of the first fluorescent signal.

13. The method of claim 10, wherein obtaining the baseline of the first fluorescent signal comprises determining a y-intercept and a baseline slope of the first fluorescent signal.

14. The method of claim 13, wherein adjusting the calculated signals each comprise matching each y-intercept of the calculated signals to the y-intercept of the first fluorescent signal and matching each baseline slope of the calculated signals to the baseline slope of the first fluorescent signal.

15. The method of claim 1, wherein each of the probes comprise tag portions hybridized to respective quenching oligonucleotide molecules at various melting temperatures.

16. The method of claim 10, wherein each of the probes comprise tag portions hybridized to respective quenching oligonucleotide molecules at various melting temperatures.
